(19) United States
(12) Patent Application Publication
(10) Pub. No.: US 2025/0342966 A1
(43) Pub. Date: Nov. 6, 2025

(54) Polygenic Risk Stratification Methods for Type 2 Diabetes

(71) Applicant: 23andMe, Inc., Sunnyvale, CA (US)

(72) Inventors: James R. Ashenhurst, San Francisco, CA (US); Bertram L. Koelsch, Salt Lake City, UT (US)

(73) Assignee: 23andMe, Inc., Sunnyvale, CA (US)

(21) Appl. No.: 18/854,208

(22) PCT Filed: Apr. 6, 2023
(86) PCT No.: PCT/US2023/017721

§ 371 Date: Oct. 4, 2024

Related U.S. Application Data
(60) Provisional application No. 63/328,656, filed on Apr. 7, 2022.

(51) Int. Cl. G16H 50/30; G16B 20/20; G16B 40/00; G16H 10/60; G16H 50/70

(52) U.S. Cl. CPC G16H 50/30; G16B 20/20; G16B 40/00; G16H 10/60; G16H 50/70

(57) ABSTRACT

The present disclosure relates to methods employing polygenic scores for determining and stratifying risk of development of type 2 diabetes mellitus (T2D) in human subjects and related prediabetes conditions such as hyperglycemia.

### CROSS-REFERENCE TO RELATED APPLICATION

This application claims priority to U.S. Provisional Application No. 63/328,656, filed Apr. 7, 2022, the contents of which are incorporated herein by reference in their entirety.

### FIELD

The present disclosure relates to methods employing polygenic scores for determining and stratifying risk of development of type 2 diabetes mellitus (T2D) in human subjects and related prediabetes conditions such as hyperglycemia.

### BACKGROUND

The United States and other Western countries face an epidemic of type 2 diabetes mellitus (T2D). Population-wide screening is critical for identifying T2D-positive and hyperglycemic individuals in order to prevent severe pathology associated with more severe or protracted disease. Despite detailed screening guidelines developed by The U.S. Preventive Services Task Force and the American Diabetes Association (ADA), diagnostic delay in prediabetes and T2D continues to hamper timely and effective treatment (Samuels et al., 2006). In 2020, the Centers for Disease Control (CDC) estimated that over 7 million undiagnosed T2D cases exist among current U.S. residents, and a diagnostic rate of only 15.3% for the 80+ million individuals living with prediabetes (Centers for Disease Control and Prevention, 2020). By 2050, the number of undiagnosed cases could be over 13 million, as T2D prevalence is projected to increase to 25-28% of the U.S. population (Boyle et al., 2010).

This high rate of progression can be mitigated with improved screening and risk stratification methods. The T2D epidemic described above is not only a case identification problem but a resource allocation problem. Novel methods are needed to improve screening and risk stratification in order to most effectively allocate resources to healthcare providers managing the prevention and treatment of the disease.

The heritability of T2D has been estimated at 25-72% (Almgren et al., 2011; Florez et al., 2018), and genome-wide association studies (GWAS) have shown a highly polygenic architecture to be associated with risk for the disease (Xue et al., 2018). Thus, predictive genetic models that produce a polygenic score (PGS) containing many thousands of genetic variants have been increasingly investigated (Reisberg et al., 2017; Khera et al., 2018).

### SUMMARY

The disclosure herein is based on a hypothesis that a T2D PGS developed from a large-scale database and consisting of over 11,000 T2D-associated genetic variants may complement existing screening methods and improve individuals' stratification across the T2D risk spectrum. First, a novel PGS was developed derived from a very large multi-ancestry sample in the applicant's database; the PGS under study herein is not the one included in the 23andMe Personal Genome Service as of January 2022. Next, the inventors hypothesized that the PGS would add unique predictive value over and above traditional factors that inform T2D screening decisions in the clinic: family history, age, and body mass index (BMI; Pippitt et al., 2016; American Diabetes Association, 2018; USPTF, 2021). It was also hypothesized that the T2D PGS would be associated with earlier age of onset of T2D, prevalence of hyperglycemia among those without a T2D diagnosis, T2D incidence after one year, and manifestations of severity including differences in T2D treatments and complications of T2D.

Previous publications have employed several methods to assess whether polygenic scores add predictive utility when used jointly with family history, including examining predictive model performance (Sun et al., 2013; Helfand, 2016; Hughes et al., 2021) and determining whether risk estimates for PGS remained significant after adjustment for family history (Tada et al., 2016).

As described in the Examples below, the T2D PGS maintained predictive utility after adjusting for family history. Combining genetics with family history led to even more improved disease risk prediction. A PGS above the 90th percentile compared to those below the 50th percentile was meaningfully related to age of onset with implications for screening practices: 18% more of those in this high genetic risk group had an age of onset prior to ages outlined in screening guidelines compared to those with below average risk. Relatedly, there was a linear and statistically significant relationship between the PGS and T2D onset (−1.3 years per standard deviation of the PGS).

Among T2D-negative individuals, the T2D PGS was associated with hyperglycemia, where each standard deviation increase of the PGS was associated with a 23% increase in the odds of hyperglycemia diagnosis. Additionally, each standard deviation increase in the PGS corresponded to a 43% increase in the odds of incident T2D at one-year follow-up.

Using complications and forms of clinical intervention (i.e., lifestyle modification, metformin treatment, or insulin treatment) as proxies for advanced illness, statistically significant associations were found between the T2D PGS and insulin treatment and diabetic neuropathy.

These findings were also replicated in a Hispanic/Latino cohort from the applicant's database, highlighting the value of the T2D PGS as a clinical tool for individuals with ancestry other than European. In this group, the T2D PGS provided additional disease risk information beyond that offered by traditional screening methodologies. The T2D PGS also had predictive value for the age of onset and for hyperglycemia among T2D-negative Hispanic/Latino participants.

These findings strengthen the notion that a T2D PGS could play a role in the clinical setting across multiple ancestries, potentially improving T2D screening practices, risk stratification, and disease management.

The disclosure herein also relates to methods of determining risk of developing type 2 diabetes (T2D) for a subject, the method comprising: (a) determining presence or absence of at least 5000 single nucleotide polymorphisms (SNPs) in a biological sample from the subject; and (b) determining a polygenic score (PGS) for the subject based on the presence or absence of the SNPs, optionally wherein each SNP is weighted by a coefficient; and (c) wherein the PGS correlates with risk of developing T2D. In some cases, the risk of developing T2D comprises risk of developing T2D within two years. In other cases, the risk of developing T2D comprises risk of developing T2D within one year. In some cases, the method further comprises determining one or more of family history of T2D, age, height, weight, and body-mass index (BMI) in the subject, wherein higher age, a BMI of at least 25 or a BMI of at least 30, and a family history of T2D each positively correlate with risk of developing T2D.

In some embodiments, the disclosure includes methods of determining risk of developing hyperglycemia for a subject, wherein the subject has not been previously diagnosed with diabetes, the method comprising: (a) determining presence or absence of at least 5000 single nucleotide polymorphisms (SNPs) in a biological sample from the subject; and (b) determining a polygenic score (PGS) for the subject based on the presence or absence of the SNPs, optionally wherein each SNP is weighted by a coefficient; and (c) wherein the PGS correlates with risk of developing hyperglycemia. In some cases, the risk of developing hyperglycemia comprises risk of developing hyperglycemia within two years. In other cases, the risk of developing hyperglycemia comprises risk of developing hyperglycemia within one year. In some cases, the method further comprises determining one or more of family history of T2D, age, height, weight, and body-mass index (BMI) in the subject, wherein higher age, a BMI of at least 25 or a BMI of at least 30, and a family history of T2D each positively correlate with risk of developing hyperglycemia.

The disclosure herein also relates to methods of analyzing the genome of a subject at risk of developing T2D, comprising: (a) determining presence or absence of at least 5000 single nucleotide polymorphisms (SNPs) in a biological sample from the subject; and (b) determining a polygenic score (PGS) for the subject based on the presence or absence of the SNPs, optionally wherein each SNP is weighted by a coefficient. In some cases, the method further comprises determining one or more of family history of T2D, age, weight, height, and body-mass index (BMI) in the subject.

In some cases, any of the above methods comprise determining presence or absence of at least 8000 SNPs; determining presence or absence of at least 10,000 SNPs; determining presence or absence of at least 11,000 SNPs; or determining presence or absence of at least 14,000 SNPs. In some cases, any of the methods above comprise determining the presence or absence of no more than 10,000 SNPs, no more than 15,000 SNPs, no more than 20,000 SNPs, or no more than 50,000 SNPs.

The present disclosure also relates to methods of treating T2D or prediabetes in a subject, the method comprising administering active surveillance to the subject, wherein the subject has been determined to be at risk of developing T2D or hyperglycemia from a process comprising: (a) determining presence or absence of at least 5000 single nucleotide polymorphisms (SNPs) in a biological sample from the subject; and (b) determining a polygenic score (PGS) for the subject based on the presence or absence of the SNPs, optionally wherein each SNP is weighted by a coefficient; and (c) wherein the PGS correlates with risk of developing T2D.

The present disclosure also relates to methods comprising administering one or more of dietary changes, insulin, metformin, thiazolidinedione, biguanide, meglitinide, DPP-4 inhibitors, sodium-glucose transporter 2 (SGLT2) inhibitor, alpha-glucosidase inhibitor, bile acid sequesters, sulfonylurea, or amylin analogs to the subject, wherein the subject has been determined to be at risk of developing T2D or hyperglycemia from a process comprising: (a) determining presence or absence of at least 5000 single nucleotide polymorphisms (SNPs) in a biological sample from the subject; and (b) determining a polygenic score (PGS) for the subject based on the presence or absence of the SNPs, optionally wherein each SNP is weighted by a coefficient; and (c) wherein the PGS correlates with risk of developing T2D. In some cases, the process further comprises determining one or more of family history of T2D, age, height, weight, and body-mass index (BMI) in the subject, wherein higher age, a BMI of at least 25 or a BMI of at least 30, and a family history of T2D each positively correlate with risk of developing T2D. In some cases, the process comprises determining presence or absence of at least 8000 SNPs. In some cases, the process comprises determining presence or absence of at least 10,000 SNPs. In some cases, the process comprises determining presence or absence of at least 11,000 SNPs. In some cases, the process comprises determining presence or absence of at least 14,000 SNPs. In some cases, the process comprises determining the presence or absence of no more than 10,000 SNPs, no more than 15,000 SNPs, no more than 20,000 SNPs, or no more than 50,000 SNPs.

In any of the above methods and processes, in some cases, the biological sample comprises genomic DNA extracted from saliva of the subject.

The disclosure herein also comprises methods of generating a polygenic score (PGS) model to determine risk of developing type 2 diabetes (T2D) or hyperglycemia in a test subject, wherein the model comprises determining the presence or absence of at least 5000 SNPs for a test subject, the method comprising: (a) receiving family history of T2D, age, and optionally height, weight, and/or BMI (“phenotypic data”) from a plurality of individuals; (b) receiving genomic DNA data from the plurality of individuals; (c) identifying a set of at least 5000 SNPs from at least one GWAS conducted in adult individuals; and (d) analyzing the genomic DNA data and the phenotypic data by regression analysis and/or machine learning to determine a set of at least 5000 SNPs that positively or negatively correlate with risk of developing T2D or hyperglycemia in the individuals, wherein the SNPs are optionally multiplied by coefficients based on the relative importance of each SNP to the risk of developing T2D or hyperglycemia. In some cases, the model comprises at least 8000 SNPs, at least 10,000 SNPs, at least 12,000 SNPs, or at least 14,000 SNPs. In some cases, the model comprises no more than 10,000 SNPs, no more than 15,000 SNPs, no more than 20,000 SNPs, or no more than 50,000 SNPs.

Methods of generating a polygenic score model that may be applicable to generation of PGS models herein are also disclosed in the applicant's US published patent application 2021/0375392, submitted May 27, 2021, which is incorporated by reference herein. Additional objects and advantages will be set forth in part in the description which follows, and in part will be understood from the description, or may be learned by practice. The objects and advantages will be realized and attained by means of the elements and combinations particularly pointed out in the appended claims. It is to be understood that both the foregoing general description and the following detailed description are exemplary and explanatory only and are not restrictive of the claims. The accompanying figures, which are incorporated in and constitute a part of this specification, serve to explain the principles described herein.

### BRIEF DESCRIPTION OF THE DRAWINGS

FIG. 1 provides a flow diagram showing participant recruitment. Three data sets were used for components of this analysis. The Descriptive Sample was used to generate plots, to estimate raw prevalences, or to estimate unadjusted odds ratios. The Incident Diagnosis Sample was used to assess the association between the polygenic score (PGS) and incident diagnosis over time. The Analytical Sample was used for regression models that included family history as a predictor. Sub-sampling was required due to missing data in key survey questions required for analysis, and participant attrition over time.

FIGS. 2A-2B show that T2D PGS is an independent predictor on par with traditional risk factors. FIG. 2A: Research participants who self-reported their family history were binarized into two groups: those with a first-degree relative with T2D and those without. The fraction of participants with a positive family history of T2D (y-axis) is plotted as a function of PGS ventile (x-axis) among T2D cases (left panel) and T2D controls (right panel). Error bars here represent empirically derived 95% confidence intervals. FIG. 2B: Unadjusted odds ratios (y-axis) of having T2D relative to the entire study population were calculated for each decade of age (left panel), BMI category (center panel), and PGS percentile (right panel). Error bars represent analytically computed 95% confidence intervals.

FIGS. 3A-3C show that T2D PGS is associated with diagnosis and incidence. FIG. 3A shows age of T2D diagnosis among cases. Mean age at T2D diagnosis (y-axis) is plotted against PGS ventiles (x-axis) among participants who self-reported their age at T2D diagnosis. FIG. 3B: Prevalence of hyperglycemia or “prediabetes” (y-axis) is plotted for T2D-negative participants against ventiles of the PGS. FIG. 3C: A one-year incidence ratio was calculated among participants who were T2D negative at an initial time point and filled out a 1-year follow-up survey. T2D incidence (y-axis) was found to increase with increasing BMI (x-axis, left panel), with age up to the 60s (x-axis, middle panel), and PGS percentile (x-axis, right panel).

FIGS. 4A-4B show that among participants with T2D, the PGS is associated with some forms of treatment and disease complications. FIG. 4A: In a dataset restricted to participants who reported a T2D diagnosis and provided information on prescribed treatments, lifestyle modifications only, metformin prescription, and insulin prescription is plotted (y-axis) for participants in the 5th, 40-60th, and 95th percentiles of the PGS (x-axis). Error bars represent empirically derived 95% confidence intervals. Lifestyle and insulin prescriptions were significantly associated with the PGS in multivariate models controlling for age, sex, BMI, and family history of T2D. FIG. 4B: Data shown are the unadjusted prevalence of complications of T2D among self-reported cases, stratified by the PGS. Error bars represent empirically derived 95% confidence intervals. Neuropathy and retinopathy were significantly associated with the PGS in multivariate models controlling for age, sex, BMI, and family history of T2D.

FIGS. 5A-5B show repeated analysis in the Hispanic/Latino sample. FIG. 5A: The prevalence of hyperglycemia among T2D-negative participants was significantly associated with the PGS, as shown with increasing ventiles of the PGS distribution. Data among T2D-positive participants are not provided due to privacy practices. FIG. 5B: Odds ratios (y-axis) of having T2D relative to the Hispanic/Latino study population were calculated for each decade of age (top panel), BMI category (center panel), and Latino-specific PGS percentile (bottom panel). Error bars represent analytically computed 95% confidence intervals. 5C: Mean age at T2D diagnosis among Hispanic/Latino (y-axis) is plotted against Hispanic/Latino-specific PGS ventiles (x-axis) among participants who self-reported their age at T2D diagnosis. Error bars represent empirically derived 95% confidence intervals.

FIG. 6 shows T2D prevalence in a multi-ancestry polygenic score training cohort.

FIG. 7 shows area under the receiver operator curve (AUC) in ancestry-specific test cohorts for the polygenic score. Demographic covariates age and sex were included in the full model but were not included in these calculations of AUC. In the bar graph, for each decade, data for females is shown to the left of data for males.

FIG. 8 shows polygenic score distributions of cases and controls in ancestry-specific test sets. In each distribution curve, the curve for cases is to the right of the curve for controls.

FIG. 9 shows calibration plots comparing the observed and estimated phenotype prevalence per ventile of the polygenic score in ancestry-specific test sets.

### DETAILED DESCRIPTION OF DESCRIPTION OF CERTAIN EMBODIMENTS

### I. Definitions

Unless otherwise defined, scientific and technical terms used in connection with the present invention shall have the meanings that are commonly understood by those of ordinary skill in the art.

In this application, the use of “or” means “and/or” unless stated otherwise. In the context of a multiple dependent claim, the use of “or” refers back to more than one preceding independent or dependent claim in the alternative only. Also, terms such as “element” or “component” encompass both elements and components comprising one unit and elements and components that comprise more than one subunit unless specifically stated otherwise.

Units, prefixes, and symbols are denoted in their Système International de Unites (SI) accepted form. Numeric ranges are inclusive of the numbers defining the range. The headings provided herein are not limitations of the various aspects of the disclosure, which can be had by reference to the specification as a whole. Accordingly, the terms defined immediately below are more fully defined by reference to the specification in its entirety.

As described herein, any concentration range, percentage range, ratio range or integer range is to be understood to include the value of any integer within the recited range and, when appropriate, fractions thereof (such as one tenth and one hundredth of an integer), unless otherwise indicated.

As utilized in accordance with the present disclosure, the following terms, unless otherwise indicated, shall be understood to have the following meanings:

The terms “correlated” and “associated” are used interchangeably herein to refer to the association between two different measurements or between a measurement or series of measurements, such as an amount or concentration of methylation in a sample or presence of a mutation, and an event, such as recurrence.

A “subject” or “individual” herein refers to a human unless explicitly stated otherwise.

The term “active surveillance,” when applied to a T2D patient or an individual at risk of developing T2D, refers to the process of monitoring such a patient by conducting regular testing to determine if insulin or another treatment such as dietary changes or other medications is needed for the patient. A “surveillance visit,” for example of a patient to a physician comprises a visit to the physician for the purpose of testing to determine if T2D is present and, if so, if further medical treatment is warranted. A patient under active surveillance may have, for example, at least one or at least two such surveillance visits per year depending on the patient's level of risk, PGS, and/or family history information.

The term “dietary changes” or the like refers to administration of changes to the diet of a subject as a means of slowing onset of T2D, treating T2D, or slowing onset of or treating symptoms of T2D, or slowing onset or treating symptoms of prediabetes, such as hyperglycemia. Dietary changes, for example, may be prescribed by a physician, in some cases through support of a nutritionist or other dietary professional.

The term “treatment” herein is broadly interpreted to include, for example, reduction of at least one symptom or complication of a disease or condition (e.g., T2D), delay of onset of a disease or condition, as well as regression or remission of a disease or condition or of at least one symptom thereof.

The term “family history,” for example, of T2D or another condition, refers to a parent and/or sibling of a subject having previously received a diagnosis of T2D or the other condition. Thus, a subject with a family history of T2D is a subject that has a parent or sibling that has previously been diagnosed with T2D.

A “body-mass index” or “BMI” is a score that is derived from a patient's weight and height. BMI is calculated as the body mass (weight) in kilograms divided by height in meters squared. A BMI of 25 kg/m² (or simply 25) or higher identifies a subject as overweight, while a BMI of 30 or higher identifies a subject as obese.

A “biological sample” herein refers to a sample taken from a subject that contains material to be analyzed, such as genomic DNA for an SNP analysis. In some cases, a biological sample may be a saliva sample, for instance.

A “GWAS” refers to a “genome-wide association study”, which is a study that tracks genomic data such as SNPs against phenotypic traits, such as diagnosis of T2D or hyperglycemia, for example.

An “SNP” refers to a “single-nucleotide polymorphism,” which refers to a substitution of a single nucleotide with a different nucleotide at a particular position in a genome, such as the human genome.

### EXAMPLES

### Example 1. Materials and Methods

### A. Study Participants and Survey Methodology

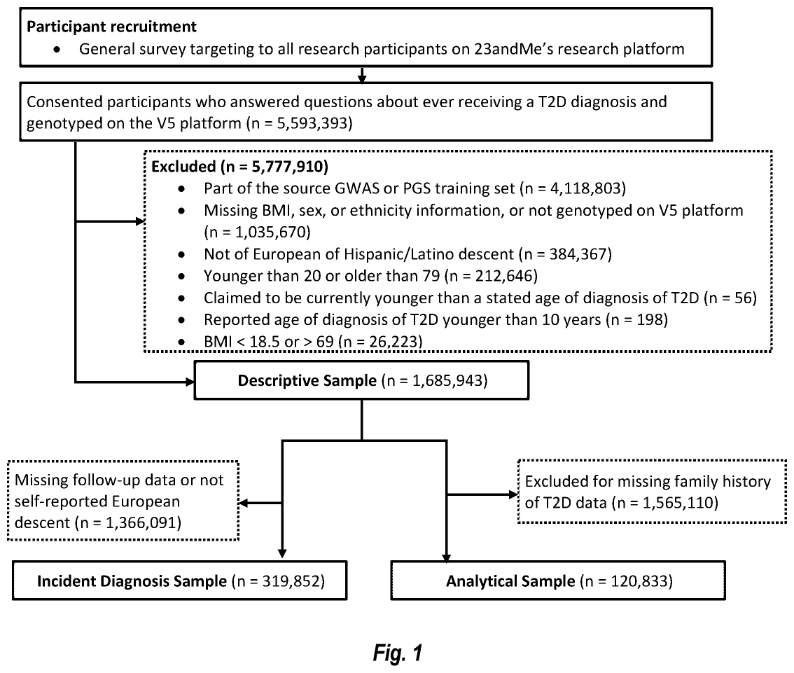

Study participants were recruited from all genotyped 23andMe customers who opted to participate in research with the applicant, 23andMe. The final Descriptive Analysis sample consisted of N=1,529,533 individuals of European descent and N=156,410 of Hispanic/Latino descent. The subsample with available family history data (the European Analytical Sample, N=113,209, Hispanic/Latino N=7,624) was smaller, as was the sample with available repeated measures (European Incidence Sample, N=319,852). Full sample descriptives are provided in Table 1, and participant exclusions are shown with a flowchart in FIG. 1.

TABLE 1
Sample Descriptives

| Self-reported | | | Sex (% | T2D |
|---|---|---|---|---|
| Ancestry | N | Age mean (SD) | Female) | Prevalence |
| European | 1,529,584 | 47.6 (15.8) | 60.4% | 3.3% |
| Hispanic/Latino | 156,410 | 41.0 (14.2) | 60.6% | 2.7% |
| European sub-sample | 113,209 | 53.3 (15.8) | 66.5% | 4.7% |
| with family history | | | | |
| data | | | | |
| Hispanic/Latino | 7,624 | 45.3 (14.9) | 64.2% | 3.8% |
| subsample with family | | | | |
| history data | | | | |
| European sub-sample | 319,852 | 50.5 (16.0) | 68.3% | 0.9% |
| with one-year | | | | |
| incidence data | | | | |

Note in Table 1 that incidence sub-sample was composed of those who were T2D-negative at baseline and provided one year follow-up data.

A series of questions asked if a participant had ever been diagnosed with T2D by a physician. Those who answered affirmatively were considered cases, whereas those who indicated no personal history of T2D were considered controls. Follow up surveys were sent annually to ascertain if any participants had newly received a diagnosis of T2D in the past 12 months. Incident cases were defined as those who had no existing diagnosis of type 2 diabetes at the baseline measurement at the time of enrollment, but who indicated a new diagnosis that occurred at least one but no more than two years after the initial question was answered. Additional questions asked about age of diagnosis of T2D, height and weight, and birth year. Ancestry category (European, Hispanic/Latino) was self-reported. Participants were required to have a minimum age of 20 and maximum age of 79 years old. Additional exclusions were: providing conceptually inconsistent responses like an age of T2D onset older than a currently reported age, reporting age of onset younger than age 10, and reporting underweight or extreme obese BMI (BMI<18.5 or >69). Individuals who were in the sample used for the GWAS or to train the PGS were excluded from the study.

Because a question from a separate survey was used to assess family history of T2D among first degree relatives, there were fewer available responses to this question relative to others, reflected in the participant flow diagram (FIG. 1). In order to maximize sample size, descriptive analyses of the data (i.e., prevalence of T2D along the spectrum of the PGS) and unadjusted odds ratios between factors like the PGS and T2D prevalence include all available data (the Descriptive Sample), whereas regression analysis involving family history were performed in a subset of the full data set with family history data (Analytical Sample). Lastly, due to loss of participation with time, the sample used to assess incidence of T2D (Incidence Sample) also represents a subset of the full data, and there was only sufficient data to perform the analysis among those of self-reported European descent (FIG. 1).

### 1. Phenotypic Definition

Cases and controls were defined based on self-reported responses to questions about current or past diagnosis of type 2 diabetes. All question phrasings focused on receiving a diagnosis of or treatment for type 2 diabetes. Although some phrasings had minor differences, the general phrasing was: “Have you ever been diagnosed with, or treated for, type 2 diabetes”. Participants answering affirmatively for either condition were treated as cases, while those who report no history of type 2 diabetes diagnosis were counted as controls. Participants who reported latent autoimmune diabetes in adults (LADA), maturity onset diabetes of the young (MODY), or only history of gestational diabetes were not counted as T2D cases. Participants who reported any history of diagnosis of “high blood sugar or prediabetes” were counted as cases of prediabetes.

Those who reported a history of T2D diagnosis were asked follow-up questions about history of prescription treatment (metformin, insulin) and physician-directed lifestyle modifications. These participants were also asked about history of diagnosis of diabetes microvascular complications: neuropathy, nephropathy, and retinopathy.

### B. Genotyping and Polygenic Score Development and Model Description

DNA extracted from saliva samples was assayed on the Illumina Infinium Global Screening Array (Illumina, San Diego, CA), consisting of approximately 640,000 common variants supplemented with ˜50,000 custom probes. This platform is referred to as 23andMe platform V5, and underwent quality controls as described previously (Nakka et al., 2019). Only participants genotyped on this platform are included in this analysis.

A polygenic score associated with the likelihood of having T2D was developed using the methods described in 23andMe White Paper 23-21 (Ashenhurst et al., 2020). Single nucleotide polymorphisms (SNPs) were selected from a meta-analysis of three GWAS conducted in individuals of European, Black/African American, and Hispanic/Latino descent.

For the development of the polygenic score (PGS), there was sufficient data to attempt ancestry-specific GWAS among those of European, Hispanic/Latino and Black/African American descent. These three GWAS were combined in a meta-analysis, from which SNP sets were selected. The GWAS meta-analysis summary statistics were pruned using p-value thresholds: 0.05, 0.005, 0.0005, and window distances: 0, 10,000, 50,000 kb.

For model training, the European, Hispanic/Latino, and Black/African American training cohorts were combined into a mega-cohort, with genetic features optimized against ancestry-specific validation sets composed of individuals of European, Hispanic/Latino, Black/African American descent. Candidate models based on nine variant sets determined by varying p-value and window distances were evaluated in tuning sets that were not included in the GWAS. Finally, based on best performance in the tuning cohorts, one variant set was chosen for final assessment in the European and Hispanic/Latino test cohorts, which were not included in the GWAS or model training.

Two variant sets were selected as the best model based on performance in the tuning sets, and were finally assessed in ancestry-specific test sets (Table 3). Additional features included in model training were age and age\hat{ }2, interactions between sex and age terms, as well as the first ten global principal components (PCs) to account for population stratification.

The final model containing 11,999 SNPs showed a significant association with the likelihood of having T2D among participants of European descent (AUC=0.656, CI [0.654,0.659], Table 3) as well as Hispanic/Latino individuals (AUC=0.635, CI [0.628,0.642]). The association was considerably higher when age and sex are also included as predictors (European AUC=0.814, CI [0.812,0.816], Hispanic/Latino AUC=0.841, CI [0.837,0.845]).

TABLE 2
Polygenic score development participant descriptive statistics

| | | | | Age | | Phenotype |
|---|---|---|---|---|---|---|
| | Genotyping | | | mean | Sex (% | prevalence |
| Sample Use | Platform | Ancestry group | N | (SD) | female) | (%) |
| GWAS | V1 to V5 | European | 1,680,210 | 48.2 | 58.7% | 6.3% |
| | | | | (15.7) | | |
| | | Hispanic/Latino | 373,496 | 40.3 | 59.1% | 5.5% |
| | | | | (13.8) | | |
| | | Sub-Saharan | 134,934 | 41.7 | 60.1% | 7.4% |
| | | African/African | | (14.4) | | |
| | | American | | | | |
| | | East/Southeast Asian | 60,530 | 37.9 | 61.0% | 3.2% |
| | | | | (12.8) | | |
| Training | V5 | European, | 2,249,141 | 46.2 | 58.9% | 6.2% |
| multi-ancestral | | Hispanic/Latino, | | (15.7) | | |
| models | | Sub-Saharan | | | | |
| | | African/African | | | | |
| | | American | | | | |
| Testing | V5 | European | 720,750 | 48.2 | 58.7% | 6.3% |
| | | | | (15.7) | | |
| | | Hispanic/Latino | 93,397 | 40.3 | 58.7% | 5.5% |
| | | | | (13.8) | | |
| | | East/Southeast Asian | 60,422 | 37.9 | 61.1% | 3.1% |
| | | | | (12.8) | | |
| | | South Asian | 32,616 | 39.8 | 42.5% | 6.1% |
| | | | | (12.7) | | |
| | | Northern | 26,542 | 42.3 | 45.5% | 3.9% |
| | | African/Western Asian | | (14.4) | | |
| | | Sub-Saharan | 15,065 | 41.7 | 60.6% | 7.2% |
| | | African/African | | (14.3) | | |
| | | American | | | | |

TABLE 3
PGS model performance metrics

| | | | Odds ratio top | |
|---|---|---|---|---|
| | Full model | Genetics only | 5% versus | Odds ratio top 5% |
| Ancestry group | AUROC | AUROC | average (95% | versus bottom 5% |
| (test sets) | (95% CIs) | (95% CIs) | CIs) | (95% CIs) |
| European | 0.8139 (0.8124 | 0.6563 (0.6539 | 3.3 (3.15 | 9.5 (8.75 |
| | to 0.8155) | to 0.6586) | to 3.39) | to 10.25) |
| Hispanic/Latino | 0.8405 (0.8365 | 0.6353 (0.6282 | 2.2 (2.0 | 10.3 (7.83 |
| | to 0.8446) | to 0.6424) | to 2.49) | to 13.5) |

Note in Table 3 that “Full model” includes the features age, age², sex, age*sex, age², and the PGS. “Genetics only” consists of only the PGS. Genome-wide principal components, which were included in training, are not included in the evaluation of model performance.

### C. Statistical Analysis

Statistical analyses were conducted in statsmodels (v0.12.1) in Python (Seabold et al., 2010). A study-wise significance threshold was defined as p<0.002 based on 26 independent comparisons and a Bonferroni correction. Reported odds ratios and linear model betas are adjusted for age, BMI (log transformed and standardized), sex, and first-degree relative family history of T2D unless otherwise described. All confidence intervals (CIs) provided are 95% CIs. To maintain participant privacy, counts or statistics that could uniquely identify fewer than five people are not provided herein.

### Example 2. The PGS is Independent of Family History

Current clinical practices rely heavily on family history of disease (FH) to identify patients at increased risk of developing conditions. But the full scope of heritability cannot be captured by FH alone, and not all individuals know their family history (e.g., those who were adopted), leaving open the possibility of under-identifying disease risk. We hypothesized that the T2D PGS combined with FH would improve the prediction of disease development more than either factor alone. This analysis was performed in the Analytical Sample (FIG. 1).

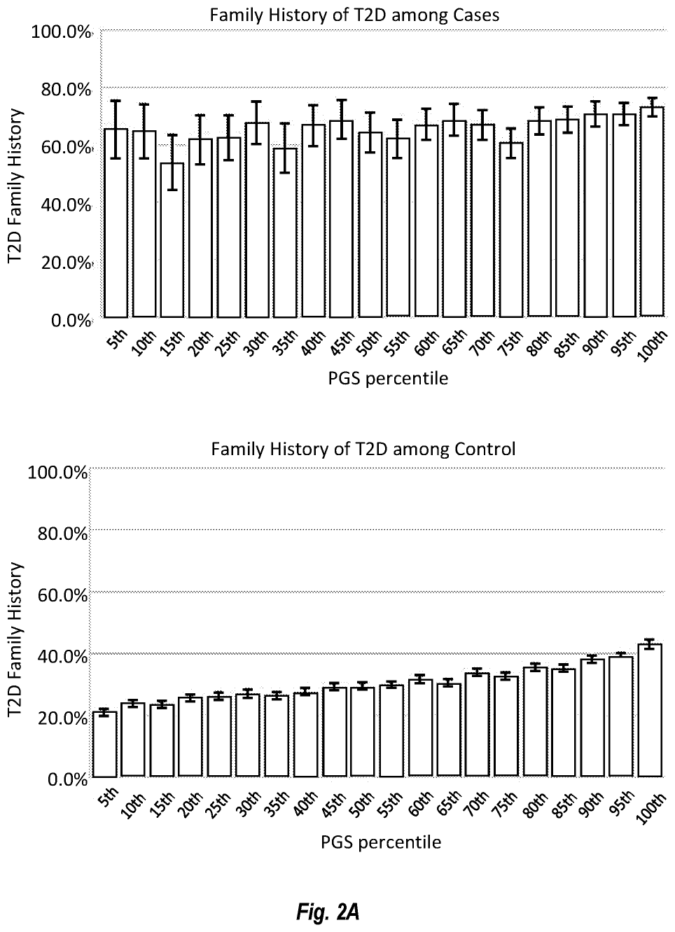

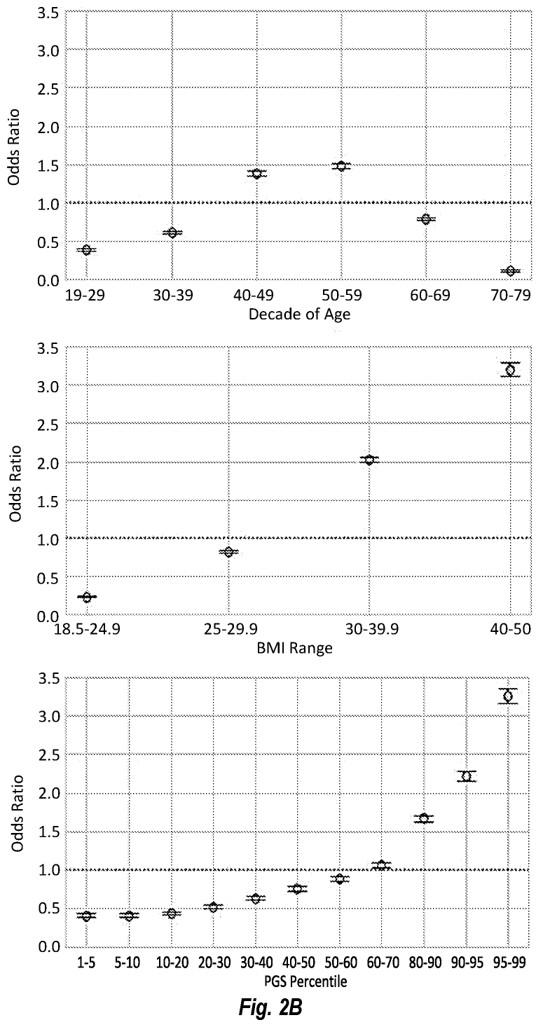

Among those in the lowest genetic risk ventile, 20.8% of controls and 65.6% of cases reported positive FH. Among those in the highest risk ventile, positive FH prevalence was 42.8% for controls and 73.1% for cases (FIG. 2A). Several logistic regression models of T2D diagnosis were assessed as a function of the T2D PGS, positive FH, and the common T2D screening factors of age and BMI (Pippitt et al., 2016; Zheng et al., 2018). Both FH and the PGS were statistically significant as independent predictors in separate models (Table 4) as well as in a model including both FH and PGS as predictors.

TABLE 4
Logistic regression between prevalent T2D, family history,
and the PGS among those of European descent

| Model (Cox-Snell's | Family History | Polygenic Score | Combined |
|---|---|---|---|
| Pseudo R2) | Only (0.18) | Only (0.18) | Model (0.22) |
| Intercept | −6.82 | −6.69 | −7.12 |
| Family History | 1.36 | — | 1.25 |
| Standardized | — | 0.48 | 0.43 |
| Polygenic Score | | | |
| Female Sex | −0.58 | −0.50 | −0.61 |
| Decade of Age | 0.058 | 0.06 | 0.06 |
| Standardized Log | 0.66 | 0.68 | 0.64 |
| Body Mass Index | | | |

Note in Table 4 that all coefficients derived from logistic regression were significant p < x in all models. N = 113,213 for all models. The model that included both family history and the PGS was the most predictive in terms of pseudo R2.

The combined model had the best predictive performance (as assessed by Cox-Snell's pseudo R2 statistic=0.22), compared to models with only FH (R2=0.18) or only the PGS (R2=0.18), showing that FH and PGS contribute unique information as predictors in each other's presence.

### Example 3. Potential Contribution of the PGS to Screening Practices

Current screening guidelines use two main sources: The U.S. Preventive Services Task Force (USPTF, 2021) and the American Diabetes Association (ADA, 2018). The USPSTF currently recommends screening for abnormal blood glucose and T2D in adults 35 to 70 years of age who are overweight or obese and repeating blood glucose testing every three years if results are typical. Individuals at higher risk should be considered for earlier screening. These risk factors include overweight and obesity, physical inactivity, abnormal lipid levels, high blood pressure, and smoking (USPSTF). The ADA proposes screening for T2D beginning at age 45 for all people. Screening for prediabetes and onset of future T2D in asymptomatic people should be considered in adults of any age who are overweight and have one or more additional risk factors for diabetes (ADA). Despite both screening recommendations, a large number of at-risk individuals, as well as prediabetic and T2D cases, are being missed annually. Thus, T2D PGS may be able to identify individuals who would benefit from earlier screening for T2D solely based on their genetic risk.

Using the T2D PGS in the Descriptive Sample, the unadjusted odds ratio (OR) of having T2D was calculated for a given PGS percentile range relative to the total population. This outcome was compared to the OR of the most common T2D risk factors of age and BMI (FIG. 2B), which were also calculated relative to the total study population. Age was scored as age of diagnosis for cases, and current age for controls. There was substantial overlap in the unadjusted OR magnitudes associated with the three variables: The range of risk associated with the PGS, OR=0.41 (CI [0.38,0.44]) at the 1st-5th percentile to OR=3.27 (CI [3.18,3.36]) at the 95th-99th percentile, was comparable to the range associated with BMI, OR=0.22 (CI [0.22,0.23]) at BMI 18.5-24.9 to OR=3.21 (CI [3.1,3.3]) at BMI 40-50. Risk of prevalent T2D was highest for ages 50-59 (OR=1.47, CI [1.44,1.51]) and lowest for ages 70-79 (OR=0.11, CI [0.10,0.12]).

Age, BMI, and the PGS were statistically significant and independent predictors of T2D prevalence in a multivariate logistic regression model described in the prior section comparing competing models (Table 2). The jointly estimated odds were as follows: decade of age (OR=1.07, CI [1.06,1.07]), log-transformed standardized BMI (OR=1.89, CI [1.84,1.94]), and the standardized PGS (OR=1.54, CI [1.51,1.58]), all ps<0.002.

Among individuals with a PGS above the 90th percentile, 49.5% of T2D-positive individuals reported age of diagnosis younger than 45 years; in contrast, 31.2% of those below the 50th percentile reported an age of diagnosis below this screening threshold, a difference of 18.3%.

### Example 4. The PGS is Associated with Age of Onset

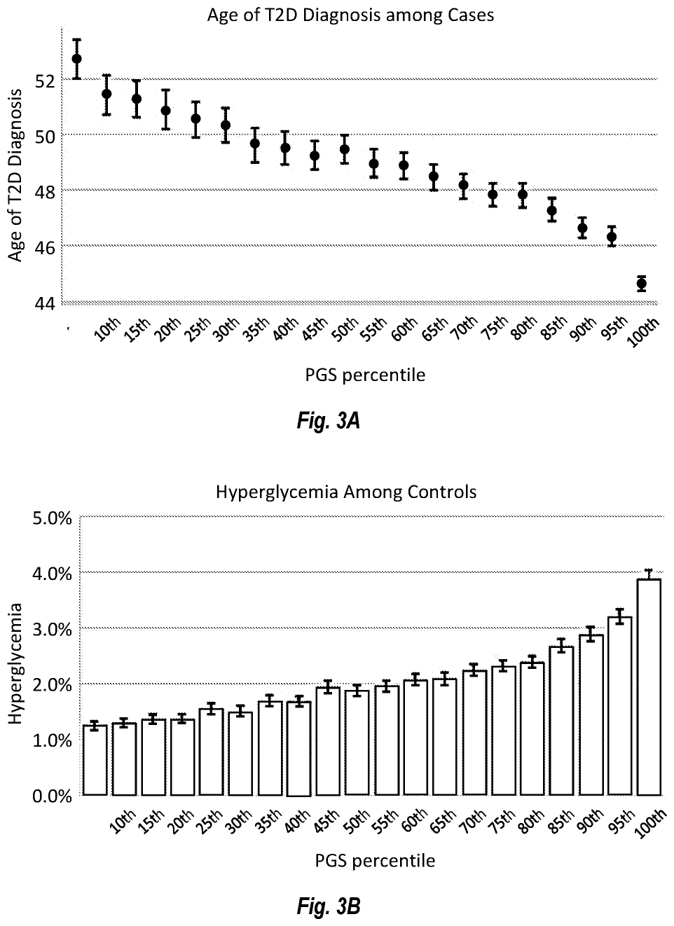

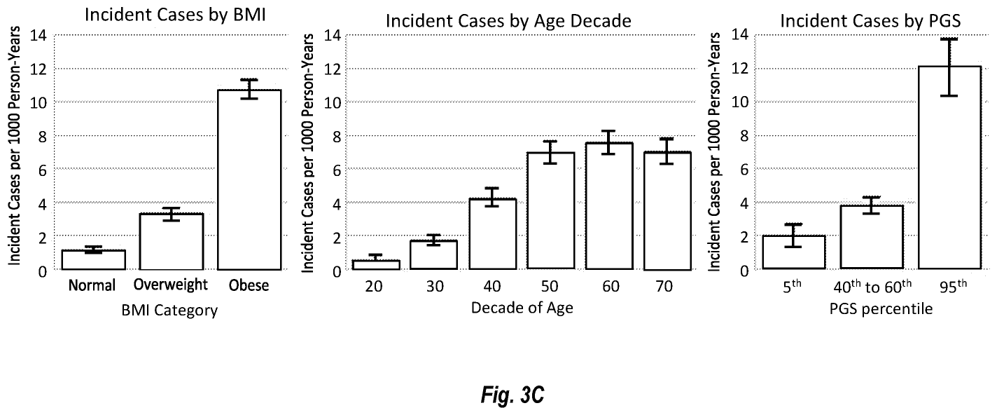

Lower age of disease onset (AOO) has been correlated with genetic risk for various conditions (Seibert et al., 2018; FinnGen et al., 2020). The relationship between the T2D PGS and self-reported T2D AOO was evaluated to assess how well the model predicts disease development timing. In the Descriptive Sample, individuals in the lowest ventile of the PGS reported a mean AOO of 52.7 years compared to 44.6 years for those in the highest ventile, a difference of 8.1 years (FIG. 3A). Furthermore, the T2D PGS was a statistically significant predictor for T2D AOO in a linear regression model that included BMI and family history of T2D in a subset of Analytic Sample who were T2D-positive and reported age of onset (N=4,745). Each standard deviation increase in the PGS was associated with a 1.34-year decrease in AOO (CI [−1.6,−1.1], p<0.002), a relationship similar to that of standardized log of BMI (B=−1.64, CI [−1.96,−1.32], p<0.002), and with positive family history of T2D (B=−1.24, CI [−1.92,−0.56], p<0.002) total model R2=0.06).

### Example 5. Hyperglycemia in T2D-Negative Individuals

The PGS model was further assessed to determine if it could also be used to predict the risk of hyperglycemia among those who were T2D-negative. Stratified by the T2D PGS, the prevalence of hyperglycemia in the highest PGS ventile in the Descriptive Sample was nearly 4-times the prevalence in the lowest PGS ventile, 1.2% vs. 3.9%, respectively (FIG. 3B). A logistic regression model of hyperglycemia diagnosis among T2D-negative individuals was evaluated using age, BMI, T2D family history, and the T2D PGS as predictors in the Analytic Sample. Each standard deviation increase of the PGS was associated with a 23% increase in the odds of hyperglycemia diagnosis (OR=1.23, CI [1.19,1.26], p<0.002). Hyperglycemia was also strongly associated with standardized log of BMI, (OR=1.60, CI [1.55,1.65], p<0.002) and family history of T2D, (OR=2.03, CI [1.89,2.18], p<0.002).

### Example 6. Incident Cases

In the subset of data with responses to annual follow-up surveys (FIG. 1; Incident Diagnosis Sample), the mean time difference between the baseline response and the follow-up response was 446 days (SD=103 days). The overall one-year incidence proportion, 4.89 per 1000 person-years, is lower than but comparable to the 6.9 per 1000 person-years statistic reported by the CDC for 2018 (Centers for Disease Control and Prevention, 2020). The incidence in the 23andMe database increased with decade of age, BMI, and PGS (FIG. 3C). Stratified by PGS, the one-year incidence of T2D in the highest genetic risk ventile was nearly six times that of individuals in the lowest ventile (2.02 vs. 12.11 cases per 1000), and roughly four times of individuals in the 40th-60th percentile (3.08 vs 12.11 cases per 1000). This rate of incidence among those with the greatest genetic risk was similar to those with obese BMI (10.71 cases per 1000 person-years).

A logistic regression model was evaluated with incident cases status as the outcome and age, standardized log BMI, T2D family history, and the PGS as predictors. The PGS proved to be a statistically significant predictor, where each standard deviation increase in PGS corresponded to a 43% increase in the odds of T2D incidence (OR=1.43, CI [1.33,1.53], p<0.002), which was about half the incident risk associated with family history (OR=3.0, CI [2.4,3.76], p<0.002), but was comparable to BMI (OR=1.82, CI [1.66,1.99], p<0.002).

### Example 7. The PGS Informs Disease Progression

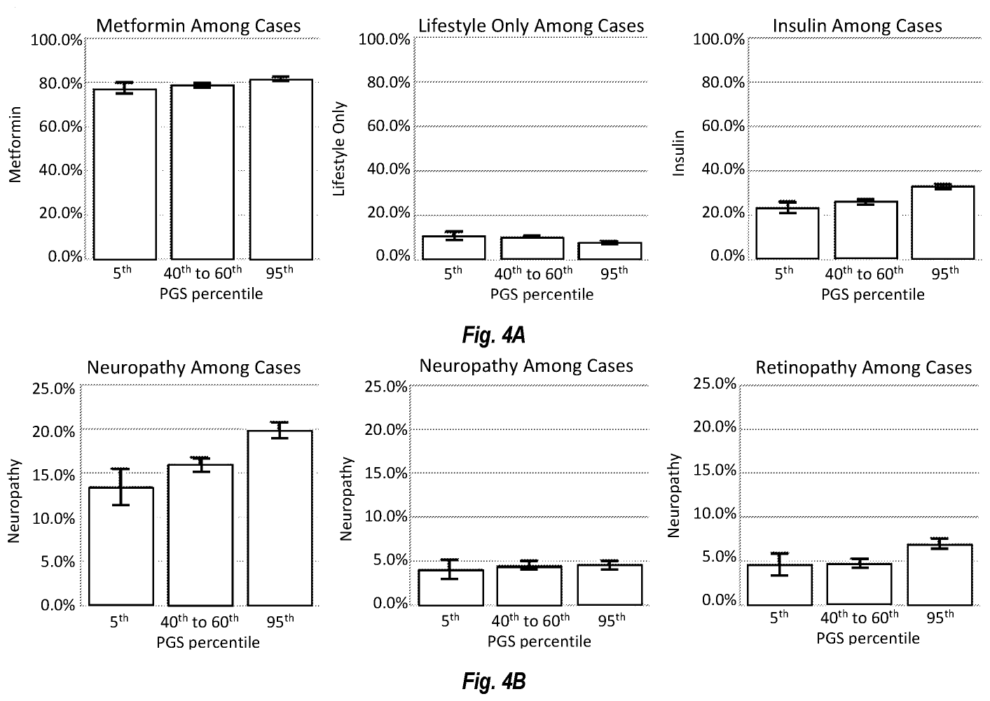

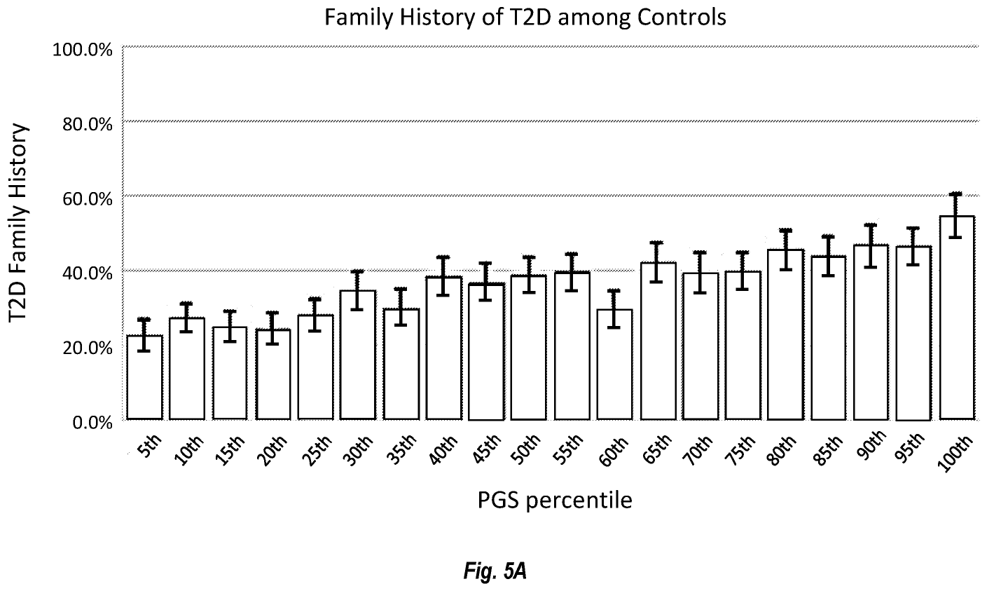

The inventors hypothesized that genetic risk for developing T2D as determined by the T2D PRS might also be associated with the risk of a more severe disease phenotype, as measured by the escalation of treatment strategy and by the prevalence of T2D complications in a cohort of T2D-positive individuals in the Analytic Sample (FIG. 1). Individuals with higher PGS values were more likely to be prescribed insulin and less likely to be following lifestyle modifications without medication (FIG. 4A). These two associations were statistically significant when the PGS was included in a logistic regression with age, sex, and BMI to predict prevalence of prescribed treatment. Each standard deviation increase in the PGS was associated with 12% lower odds of following only lifestyle modifications, but this was not statistically significant when accounting for the study-wise correction, (OR=0.89, CI [0.82,0.96], p=0.004), and 14% higher odds of being prescribed insulin (OR=1.14, CI [1.09,1.19], p<0.002). The PGS was not a statistically significant predictor of metformin treatment.

This approach was repeated to assess the utility of the PGS for predicting complications of poorly-controlled T2D (FIG. 4B). Each standard deviation increase in the PGS was associated with 13% higher odds of diabetic neuropathy (OR=1.13, CI [1.07,1.19], p<0.002). However, the PGS was not statistically significantly associated with higher odds of diabetic nephropathy (OR=1.11, CI [1.01,1.22], p=0.02) or with diabetic retinopathy (OR=1.13, CI [1.03,1.23], p=0.007). Together, these data show the T2D PGS is associated with some but not all forms of disease severity as measured by prescribed treatment and prevalence of complications.

### Example 8. PGS Associations are Transferable to Hispanic/Latino Individuals

The inventors next determined whether findings showing the relevance of the T2D PGS would replicate in other ethnicities by testing a self-reported 23andMe Hispanic/Latino cohort (N=156,410, see Methods and Materials and FIG. 1 for participant recruitment flowchart).

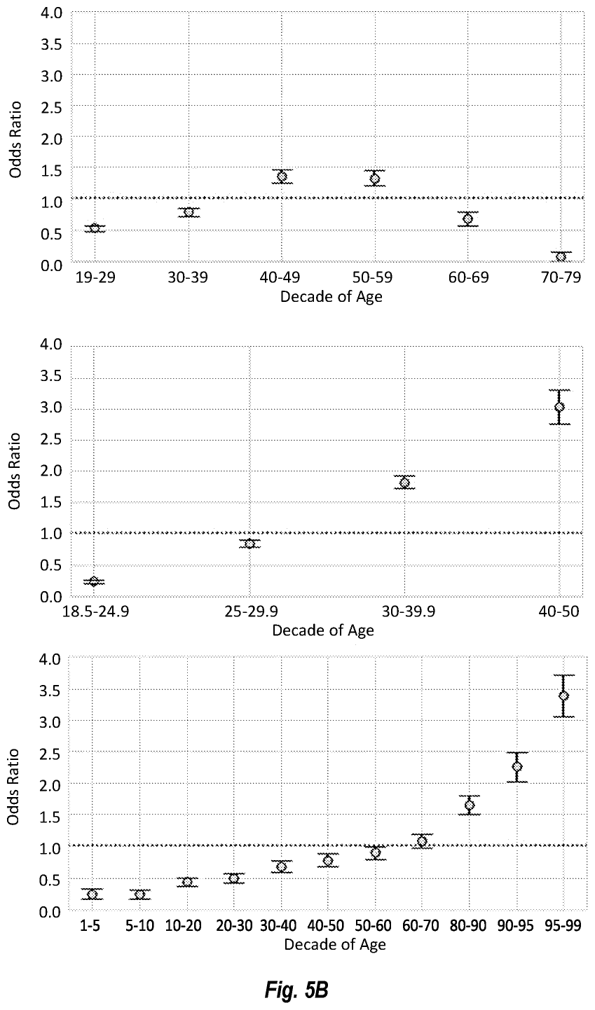

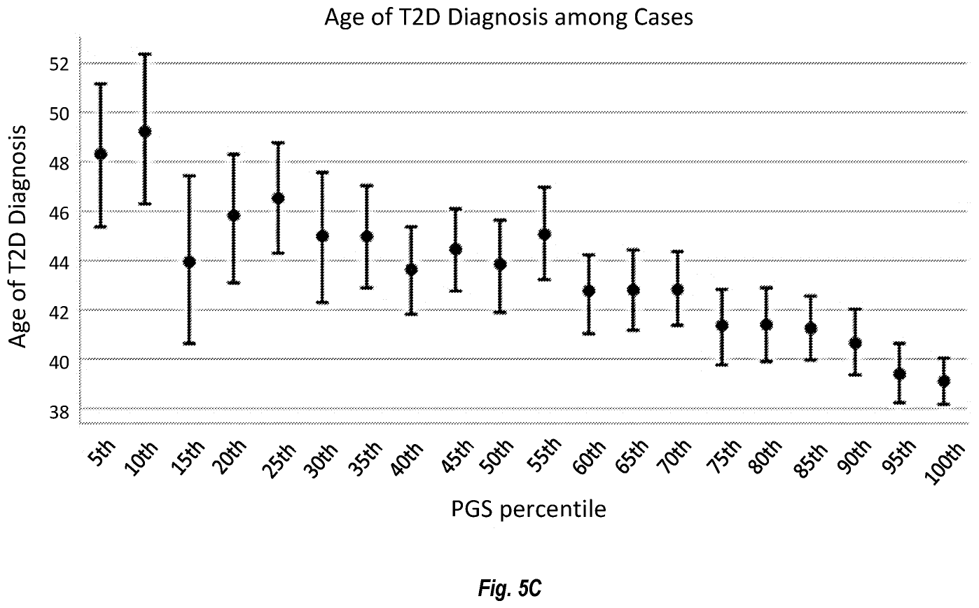

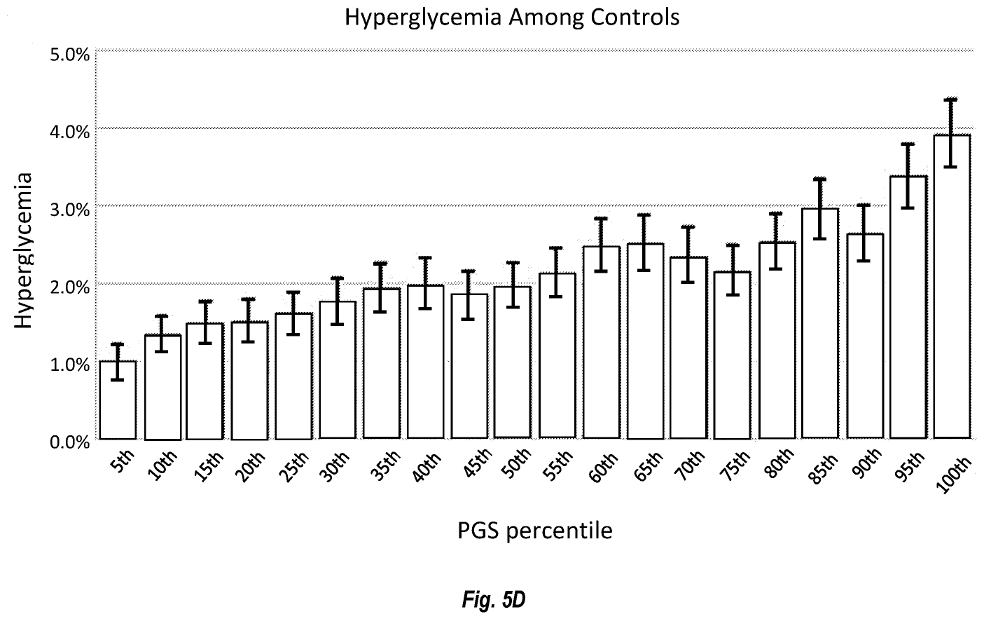

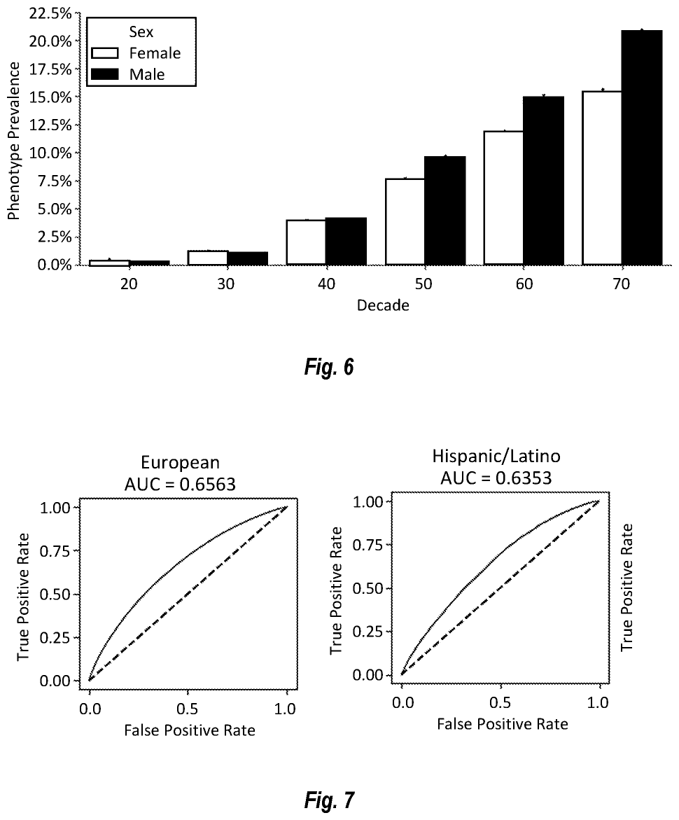

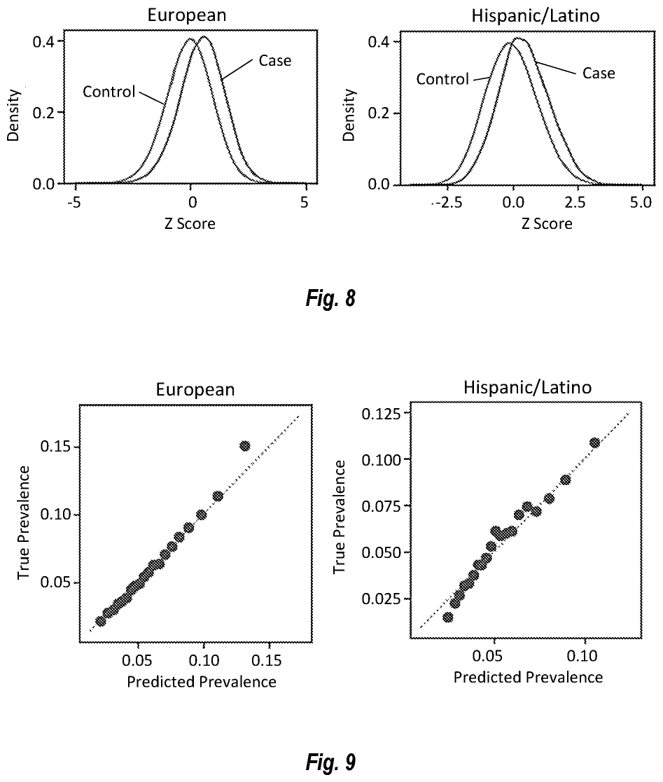

Among those who were T2D-negative at the time of the survey, family history of T2D was more common among those with higher genetic risk as indexed by the PGS than lower (FIG. 5A; data for T2D-positive cases not shown due to smaller sample size and privacy requirements). The PGS performance was examined as a predictor of T2D while controlling for T2D family history. This analysis showed the PGS to be an independent and statistically significant predictor of T2D in a model containing age, BMI, family history, and the PGS (OR=1.51, CI [1.37,1.66], p<0.002; Table 5).

TABLE 5
Logistic regression between prevalent T2D, family history,
and the PGS in the Hispanic/Latino replication sample

| Model (Cox-Snell's | Family History | Polygenic Score | Combined |
|---|---|---|---|
| Pseudo R2) | Only (0.20) | Only (0.18) | Model (0.23) |
| Intercept | −6.74 | −6.63 | −7.01 |
| Family History | 1.73 | — | 1.61 |
| Standardized | — | 0.47 | 0.41 |
| Polygenic Score | | | |
| Female Sex | −0.51 | −0.44 | −0.52 |
| Decade of Age | 0.05 | 0.06 | 0.06 |
| Standardized Log | 0.69 | 0.70 | 0.66 |
| Body Mass Index | | | |

Note in Table 5 that all coefficients derived from logistic regression were significant p < x in all models. N = 7,624 for all models. The model that included both family history and the PGS was the most predictive in terms of pseudo R2.

The PGS's ability to stratify Hispanic/Latino individuals by an unadjusted odds ratio of having T2D as compared to age and BMI (FIG. 5B) was also examined. Similar trends were observed as reported in the European cohort; the range of risk associated with the PGS, OR=0.24 (CI [0.18,0.32]) at the 1st-5th percentile to OR=3.38 (CI [3.09,3.71]) at the 95th-99th percentile, was comparable to the range associated with BMI, OR=0.23 (CI [0.20,0.26]) at BMI 18.5 to 24.9 to OR=3.03 (CI [2.78,3.32]) at BMI 40-50. Risk of prevalent T2D was highest for ages 40-49 (OR=1.35, CI [1.25,1.46]) and lowest for ages 70-79 (OR=0.07, CI [0.04,0.14]).

Among individuals with PGS above the 90th percentile, 56.4% of Hispanic/Latino T2D-positive individuals reported an age of diagnosis younger than 40 years. In contrast, 36.4% of those below the 50th percentile reported an age of diagnosis below this screening threshold, a difference of 20%.

There was a correlation between increasing PGS and younger age of T2D onset in the Latino cohort (FIG. 5C). Mean AOO ranged from 48.3 to 39.1 years from lowest to highest PGS ventile, a difference of 9.2 years. However, this relationship was not statistically significant (β=−0.60, CI [−1.66,0.47], p=0.27) in a linear model trained to predict AOO from BMI, family history of T2D, and genetics in a small subset of the Hispanic/Latino cohort with complete data (N=256).

Hyperglycemia in Hispanic/Latino T2D controls was more prevalent in those with higher PGS. In the highest PGS ventile was also nearly 4-times the prevalence in the lowest PGS ventile, 1.0% vs. 3.9%, respectively (FIG. 5D). A logistic regression model of hyperglycemia diagnosis among T2D-negative individuals was examined using age, BMI, T2D family history, and the T2D PGS as predictors. One standard deviation in the PGS was associated with a 35% increase in the odds of hyperglycemia among those without T2D (OR=1.35, CI [1.22,1.51], p<0.002), which was comparable to that of standardized log-BMI (OR=1.65, CI [1.46,1.86], p<0.002) and family history of T2D (OR=1.60, CI [1.22,2.11], p<0.002).

Insufficient data were available in the Hispanic/Latino population to evaluate the association between the T2D PGS and incident diagnosis, treatment prevalences, or disease complications.

### Example 9. Discussion of Examples 1-8

Examples 1-8 demonstrate the utility of the PGS in identifying individuals with increased risk for hyperglycemia among the T2D-negative population. Furthermore, the PGS was also highly correlated with earlier age of T2D onset, and can be used to predict incident T2D cases from a population of susceptible individuals. The risk profile conferred by increasing PGS was comparable to risk associated with increasing age and BMI. Taken together, these findings argue strongly for including the T2D PGS in a clinical assessment of T2D risk and prophylactic decision-making.

The present study found an increasing relationship between T2D genetic risk and positive family history among European-descent and Hispanic/Latino-descent T2D-negative individuals. There was also, however, a substantial number of cases and controls in each population who present with negative family history despite high genetic risk, suggesting that family history is not equivalent to genetic risk. Factors other than genetics, such as common environment, may also contribute to the risk conferred by family history. Ultimately, a model including both family history and the PGS proved better at predicting T2D than each factor separately.

Screening for hyperglycemia and T2D has been based on a set of guidelines that determine eligibility based on well-documented risk factors such as age, BMI, positive family history, membership in a high-risk race or ethnic group, and environmental factors (Pippitt et al., 2016). The present study demonstrated the validity of the T2D PGS as an independent risk factor for hyperglycemia. The results reveal that a meaningful number of genetically high-risk individuals would not qualify for screening under the current criteria. The inclusion of the PGS as a risk factor could, therefore, improve the efficacy of screening programs, especially as costs for genome-wide genotyping continue to decrease. The impact of screening individuals with a PGS above the 90th percentile compared to those below the 50th percentile showed that approximately 20% more of those in the 90th percentile had an age of onset prior to ages outlined in screening guidelines compared to those in the 50th percentile. Thus, these high genetic risk individuals could benefit from earlier screening.

The present study found T2D PGS to correlate with treatment options, where those at lower PGS were more likely to be prescribed lifestyle modifications whereas those at higher PGS were more likely to be treated with insulin. Metformin treatment was not associated with the PGS. For complications of T2D, the PGS correlated with some (e.g., neuropathy) but not others (e.g., nephropathy and retinopathy).

There is a Euro-centric bias in the field of polygenic risk prediction (Martin et al., 2019). Thus, the PGS was also tested in the 23andMe Hispanic/Latino cohort and was found to have roughly comparable performance in this group as in European-descent individuals, as evidenced by the AUROC (0.656 in European-descent and 0.635 in Hispanic/Latino-descent individuals) and other risk stratification statistics. Furthermore, this cohort had sufficient family history and incidence data for a sufficiently powered study. Our analyses show that, as in the European cohort, the PGS provided valuable information for identifying at-risk Hispanic/Latino individuals, on par with risk factors already used for clinical decision-making. These findings serve as an important proof of principle for the application of polygenic prediction to assessing risk in underserved populations.

The present study has several limitations that should be considered when interpreting the results. All phenotypes were obtained through participant self-report, although previous work by the applicant has shown the accuracy and robustness of this form of data collection at scale (Eriksson et al., 2010; Tung et al., 2011). Missing data across survey instruments resulted in smaller subsamples used for regression modeling compared to the larger sample with T2D diagnostic and demographic information. Models assumed linear relationships between the outcomes and age or BMI, whereas non-linear relationships may better explain the data.

### REFERENCES

23andMe (2019). 23andMe's Populations Collaborations Program Supports Research in Understudied Groups. 23andMe Blog. Available at: https://blog.23andme.com/23andme-research/23andmes-population-collaboration-program-supports-research-in-understudied-groups/[Accessed Jul. 24, 2020].
Almgren, P., Lehtovirta, M., Isomaa, B., Sarelin, L., Taskinen, M. R., Lyssenko, V., et al. (2011). Heritability and familiality of type 2 diabetes and related quantitative traits in the Botnia Study. Diabetologia 54, 2811-2819. doi: 10.1007/s00125-011-2267-5.
American Diabetes Association (2018). 2. Classification and Diagnosis of Diabetes: Standards of Medical Care in Diabetes-2018. Diabetes Care 41, S13-S27. doi: 10.2337/dc18-S002.
Ashenhurst, J. R., Zhan, J., Multhaup, M. L., Kita, R., Sazonova, O. V., Krock, B., et al. (2020). A Generalized Method for the Creation and Evaluation of Polygenic Scores. 23andMe, Inc. Available at: https://permalinks.23andme.com/pdf/23_21-PRSMethodology_May2020.pdf [Accessed Jan. 3, 2022].
Boyle, J. P., Thompson, T. J., Gregg, E. W., Barker, L. E., and Williamson, D. F. (2010). Projection of the year 2050 burden of diabetes in the US adult population: dynamic modeling of incidence, mortality, and prediabetes prevalence. Popul Health Metr 8, 29. doi: 10.1186/1478-7954-8-29.
Centers for Disease Control and Prevention (2020). National Diabetes Statistics Report. Atlanta (GA): U.S. Dept of Health and Human Services Available at: https://www.cdc.gov/diabetes/pdfs/data/statistics/national-diabetes-statistics-report.pdf.
Chen, L., Wang, Y.-F., Liu, L., Bielowka, A., Ahmed, R., Zhang, H., et al. (2020). Genome-wide assessment of genetic risk for systemic lupus erythematosus and disease severity. Hum. Mol. Genet. 29, 1745-1756. doi: 10.1093/hmg/ddaa030.
Eriksson, N., Macpherson, J. M., Tung, J. Y., Hon, L. S., Naughton, B., Saxonov, S., et al. (2010). Web-based, participant-driven studies yield novel genetic associations for common traits. PLOS Genet. 6, e1000993. doi: 10.1371/journal.pgen. 1000993.
FinnGen, Mars, N., Koskela, J. T., Ripatti, P., Kiiskinen, T. T. J., Havulinna, A. S., et al. (2020). Polygenic and clinical risk scores and their impact on age at onset and prediction of cardiometabolic diseases and common cancers. Nature Medicine 26, 549-557. doi: 10.1038/s41591-020-0800-0.
Florez, J. C., Udler, M. S., and Hanson, R. L. (2018). “Genetics of Type 2 Diabetes,” in Diabetes in America, eds. C. C. Cowie, S. S. Casagrande, A. Menke, M. A. Cissell, M. S. Eberhardt, J. B. Meigs, et al. (Bethesda (MD): National Institute of Diabetes and Digestive and Kidney Diseases (US)). Available at: http://www.ncbi.nlm.nih.gov/books/NBK567998/[Accessed Jan. 28, 2022].
Glechner, A., Keuchel, L., Affengruber, L., Titscher, V., Sommer, I., Matyas, N., et al. (2018). Effects of lifestyle changes on adults with prediabetes: A systematic review and meta-analysis. Prim Care Diabetes 12, 393-408. doi: 10.1016/j.pcd.2018.07.003.
Helfand, B. T. (2016). A comparison of genetic risk score with family history for estimating prostate cancer risk. Asian J Androl 18, 515-519. doi: 10.4103/1008-682X.177122.
Hughes, E., Tshiaba, P., Wagner, S., Judkins, T., Rosenthal, E., Roa, B., et al. (2021). Integrating Clinical and Polygenic Factors to Predict Breast Cancer Risk in Women Undergoing Genetic Testing. JCO Precis Oncol 5, PO.20.00246. doi: 10.1200/PO.20.00246.
Khera, A. V., Chaffin, M., Aragam, K. G., Haas, M. E., Roselli, C., Choi, S. H., et al. (2018). Genome-wide polygenic scores for common diseases identify individuals with risk equivalent to monogenic mutations. Nat. Genet. 50, 1219-1224. doi: 10.1038/s41588-018-0183-z.
Martin, A. R., Kanai, M., Kamatani, Y., Okada, Y., Neale, B. M., and Daly, M. J. (2019). Clinical use of current polygenic risk scores may exacerbate health disparities. Nat. Genet. 51, 584-591. doi: 10.1038/s41588-019-0379-x.
Nakka, P., Pattillo Smith, S., O'Donnell-Luria, A. H., McManus, K. F., Mountain, J. L., Ramachandran, S., et al. (2019). Characterization of Prevalence and Health Consequences of Uniparental Disomy in Four Million Individuals from the General Population. The American Journal of Human Genetics 105, 921-932. doi: 10.1016/j.ajhg.2019.09.016.
Oetjens, M. T., Kelly, M. A., Sturm, A. C., Martin, C. L., and Ledbetter, D. H. (2019). Quantifying the polygenic contribution to variable expressivity in eleven rare genetic disorders. Nat Commun 10, 4897. doi: 10.1038/s41467-019-12869-0.
Paul, K. C., Schulz, J., Bronstein, J. M., Lill, C. M., and Ritz, B. R. (2018). Association of Polygenic Risk Score With Cognitive Decline and Motor Progression in Parkinson Disease. JAMA Neurology. doi: 10.1001/jamaneurol.2017.4206.
Pippitt, K., Li, M., and Gurgle, H. E. (2016). Diabetes Mellitus: Screening and Diagnosis. AFP 93, 103-109.
Reisberg, S., Iljasenko, T., Läll, K., Fischer, K., and Vilo, J. (2017). Comparing distributions of polygenic risk scores of type 2 diabetes and coronary heart disease within different populations. PloS one 12, e0179238.
Samuels, T. A., Cohen, D., Brancati, F. L., Coresh, J., and Kao, W. H. L. (2006). Delayed diagnosis of incident type 2 diabetes mellitus in the ARIC study. Am J Manag Care 12, 717-724.
Seabold, Skipper, and Perktold, J. (2010). statsmodels: Economic and statistical modeling with python. Proceedings of the 9th Python in Science Conference.
Seibert, T. M., Fan, C. C., Wang, Y., Zuber, V., Karunamuni, R., Parsons, J. K., et al. (2018). Polygenic hazard score to guide screening for aggressive prostate cancer: development and validation in large scale cohorts. BMJ, j5757. doi: 10.1136/bmj.j5757.
Sun, J., Na, R., Hsu, F.-C., Zheng, S. L., Wiklund, F., Condreay, L. D., et al. (2013). Genetic score is an objective and better measurement of inherited risk of prostate cancer than family history. Eur Urol 63, 585-587. doi: 10.1016/j.eururo.2012.11.047.
Tabák, A. G., Herder, C., Rathmann, W., Brunner, E. J., and Kivimäki, M. (2012). Prediabetes: a high-risk state for diabetes development. Lancet 379, 2279-2290. doi: 10.1016/S0140-6736 (12) 60283-9.
Tada, H., Melander, O., Louie, J. Z., Catanese, J. J., Rowland, C. M., Devlin, J. J., et al. (2016). Risk prediction by genetic risk scores for coronary heart disease is independent of self-reported family history. Eur Heart J 37, 561-567. doi: 10.1093/eurheartj/ehv462.
Tremblay, J., Haloui, M., Attaoua, R., Tahir, R., Hishmih, C., Harvey, F., et al. (2021). Polygenic risk scores predict diabetes complications and their response to intensive blood pressure and glucose control. Diabetologia 64, 2012-2025. doi: 10.1007/s00125-021-05491-7.
Tung, J. Y., Do, C. B., Hinds, D. A., Kiefer, A. K., Macpherson, J. M., Chowdry, A. B., et al. (2011). Efficient replication of over 180 genetic associations with self-reported medical data. PLOS ONE 6, e23473. doi: 10.1371/journal.pone.0023473.
Udler, M. S., Kim, J., von Grotthuss, M., Bonàs-Guarch, S., Cole, J. B., Chiou, J., et al. (2018). Type 2 diabetes genetic loci informed by multi-trait associations point to disease mechanisms and subtypes: A soft clustering analysis. PLOS Med 15, e1002654. doi: 10.1371/journal.pmed.1002654.
USPTF (2021). Prediabetes and Type 2 Diabetes: Screening. US Preventive Service Task Force. Available at: https://www.uspreventiveservicestaskforce.org/uspstf/recommendation/screening-for-prediabetes-and-type-2-diabetes [Accessed Jan. 4, 2022].
Xue, A., Wu, Y., Zhu, Z., Zhang, F., Kemper, K. E., Zheng, Z., et al. (2018). Genome-wide association analyses identify 143 risk variants and putative regulatory mechanisms for type 2 diabetes. Nat Commun 9, 2941. doi: 10.1038/s41467-018-04951-w.
Zheng, Y., Ley, S. H., and Hu, F. B. (2018). Global aetiology and epidemiology of type 2 diabetes mellitus and its complications. Nature Reviews Endocrinology 14, 88-98. doi: 10.1038/nrendo.2017.151.

The foregoing written disclosure is considered to be sufficient to enable one skilled in the art to practice the embodiments. The foregoing description and Examples detail certain embodiments and describes the best mode contemplated by the inventors. It will be appreciated, however, that no matter how detailed the foregoing may appear in text, the embodiment may be practiced in many ways and should be construed in accordance with the appended claims and any equivalents thereof. All references cited herein are incorporated by reference in their entirety.

What is claimed is:

1. A method of determining risk of developing type 2 diabetes (T2D) for a subject, the method comprising:
a. determining presence or absence of at least 5000 single nucleotide polymorphisms (SNPs) in a biological sample from the subject; and
b. determining a polygenic score (PGS) for the subject based on the presence or absence of the SNPs, optionally wherein each SNP is weighted by a coefficient;
c. wherein the PGS correlates with risk of developing T2D.

2. The method of claim 1, wherein the risk of developing T2D comprises risk of developing T2D within two years.

3. The method of claim 1, wherein the risk of developing T2D comprises risk of developing T2D within one year.

4. The method of any one of claims 1-3, wherein the method further comprises determining one or more of family history of T2D, age, height, weight, and body-mass index (BMI) in the subject, wherein higher age, a BMI of at least 25 or a BMI of at least 30, and a family history of T2D each positively correlate with risk of developing T2D.

5. A method of determining risk of developing hyperglycemia for a subject, wherein the subject has not been previously diagnosed with diabetes, the method comprising:
a. determining presence or absence of at least 5000 single nucleotide polymorphisms (SNPs) in a biological sample from the subject; and
b. determining a polygenic score (PGS) for the subject based on the presence or absence of the SNPs, optionally wherein each SNP is weighted by a coefficient;
c. wherein the PGS correlates with risk of developing hyperglycemia.

6. The method of claim 5, wherein the risk of developing hyperglycemia comprises risk of developing hyperglycemia within two years.

7. The method of claim 5, wherein the risk of developing hyperglycemia comprises risk of developing hyperglycemia within one year.

8. The method of any one of claims 5-7, wherein the method further comprises determining one or more of family history of T2D, age, height, weight, and body-mass index (BMI) in the subject, wherein higher age, a BMI of at least 25 or a BMI of at least 30, and a family history of T2D each positively correlate with risk of developing hyperglycemia.

9. A method of analyzing the genome of a subject at risk of developing T2D, comprising:
a. determining presence or absence of at least 5000 single nucleotide polymorphisms (SNPs) in a biological sample from the subject; and
b. determining a polygenic score (PGS) for the subject based on the presence or absence of the SNPs, optionally wherein each SNP is weighted by a coefficient.

10. The method of claim 9, wherein the method further comprises determining one or more of family history of T2D, age, weight, height, and body-mass index (BMI) in the subject.

11. The method of any one of claims 1-10, wherein the method comprises determining presence or absence of at least 8000 SNPs.

12. The method of any one of claims 1-10, wherein the method comprises determining presence or absence of at least 10,000 SNPs.

13. The method of any one of claims 1-10, wherein the method comprises determining presence or absence of at least 11,000 SNPs.

14. The method of any one of claims 1-10, wherein the method comprises determining presence or absence of at least 14,000 SNPs.

15. The method of any one of claims 1-14, wherein the method comprises determining the presence or absence of no more than 10,000 SNPs, no more than 15,000 SNPs, no more than 20,000 SNPs, or no more than 50,000 SNPs.

16. A method of treating T2D or prediabetes in a subject, the method comprising administering active surveillance to the subject, wherein the subject has been determined to be at risk of developing T2D or hyperglycemia from a process comprising:
a. determining presence or absence of at least 5000 single nucleotide polymorphisms (SNPs) in a biological sample from the subject; and
b. determining a polygenic score (PGS) for the subject based on the presence or absence of the SNPs, optionally wherein each SNP is weighted by a coefficient;
c. wherein the PGS correlates with risk of developing T2D.

17. A method of treating T2D in a subject, the method comprising administering one or more of dietary changes, insulin, metformin, thiazolidinedione, biguanide, meglitinide, DPP-4 inhibitors, sodium-glucose transporter 2 (SGLT2) inhibitor, alpha-glucosidase inhibitor, bile acid sequesters, sulfonylurea, or amylin analogs to the subject, wherein the subject has been determined to be at risk of developing T2D or hyperglycemia from a process comprising:
a. determining presence or absence of at least 5000 single nucleotide polymorphisms (SNPs) in a biological sample from the subject; and
b. determining a polygenic score (PGS) for the subject based on the presence or absence of the SNPs, optionally wherein each SNP is weighted by a coefficient;
c. wherein the PGS correlates with risk of developing T2D.

18. The method of claim 16 or 17, wherein the process further comprises determining one or more of family history of T2D, age, height, weight, and body-mass index (BMI) in the subject, wherein higher age, a BMI of at least 25 or a BMI of at least 30, and a family history of T2D each positively correlate with risk of developing T2D.

19. The method of any one of claims 16-18, wherein the process comprises determining presence or absence of at least 8000 SNPs.

20. The method of any one of claims 16-18, wherein the process comprises determining presence or absence of at least 10,000 SNPs.

21. The method of any one of claims 16-18, wherein the process comprises determining presence or absence of at least 11,000 SNPs.

22. The method of any one of claims 16-18, wherein the process comprises determining presence or absence of at least 14,000 SNPs.

23. The method of any one of claims 16-18, wherein the process comprises determining the presence or absence of no more than 10,000 SNPs, no more than 15,000 SNPs, no more than 20,000 SNPs, or no more than 50,000 SNPs.

24. The method of any one of claims 1-23, wherein the biological sample comprises genomic DNA extracted from saliva of the subject.

25. A method of generating a polygenic score (PGS) model to determine risk of developing type 2 diabetes (T2D) or hyperglycemia in a test subject, wherein the model comprises determining the presence or absence of at least 5000 SNPs for a test subject, the method comprising:
a. receiving family history of T2D, age, and optionally height, weight, and/or BMI (“phenotypic data”) from a plurality of individuals;
b. receiving genomic DNA data from the plurality of individuals;
c. identifying a set of at least 5000 SNPs from at least one GWAS conducted in adult individuals; and
d. analyzing the genomic DNA data and the phenotypic data by regression analysis and/or machine learning to determine a set of at least 5000 SNPs that positively or negatively correlate with risk of developing T2D or hyperglycemia in the individuals, wherein the SNPs are optionally multiplied by coefficients based on the relative importance of each SNP to the risk of developing T2D or hyperglycemia.

26. The method of claim 25, wherein the model comprises at least 8000 SNPs.

27. The method of claim 25, wherein the model comprises at least 10,000 SNPs.

28. The method of claim 25, wherein the model comprises at least 12,000 SNPs.

29. The method of claim 25, wherein the model comprises at least 14,000 SNPs.

30. The method of any one of claims 25-29, wherein the model comprises no more than 10,000 SNPs, no more than 15,000 SNPs, no more than 20,000 SNPs, or no more than 50,000 SNPs.
